(19) United States
(12) Patent Application Publication
(10) Pub. No.: US 2020/0339929 A1
(43) Pub. Date: Oct. 29, 2020

(54) CELL MOUNTING MODULE DEVICE

(71) Applicant: BIOT KOREA INC., Gwangju (KR)

(72) Inventors: Yeong Jun CHANG, Seoul (KR); Sung Kuk PARK, Seoul (KR); Tae Yong KIM, Gwangju (KR)

(73) Assignee: Biot Korea Inc., Gwangju (KR)

(21) Appl. No.: 16/621,343

(22) PCT Filed: Sep. 9, 2019
(86) PCT No.: PCT/KR2019/011608

§ 371 Date: Dec. 11, 2019

(30) Foreign Application Priority Data
Dec. 11, 2018 (KR) 10-2018-0159312

(51) Int. Cl. C12M 1/12; A61L 27/56; A61L 27/58; A61B 10/00; A61L 27/38

(52) U.S. Cl. CPC C12M 25/14; A61L 27/56; A61L 27/38; A61B 10/0096; A61L 27/58

(57) ABSTRACT

The present invention relates to a cell mounting module device, which comprises a body portion having a container form of which upper and lower portions are opened and an inside is empty, a cap portion which includes an upper cap and a lower cap coupled to opened upper and lower ends of the upper and lower portions of the body portion to be opened and closed, respectively, and has a syringe coupling member formed at a center of the lower cap, a cell accommodating portion which is installed in the body portion and accommodates cells injected through the opened upper portion of the body portion, and a cell mounding module portion which is installed below the cell accommodating portion and includes a microstructure for mounting the cells accommodated in the cell accommodating portion.

### TECHNICAL FIELD

The present invention relates to a cell mounting module technology, and more particularly, to a cell mounting module device which is fabricated in a small module type to increase cell mounting and recovery efficiency and exclude a possibility of cross infection for a microstructure.

### BACKGROUND ART

Stem cells are actually referred to as an embryonic pluripotent cells. The stem cells means cells which may be developed to any tissue. The stem cells are primarily extracted from embryos in an early division stage. Since the cells in this stage yet have no organ forming ability, the cells may be cultured in a cell line specifically selected according to a pre-input.

Currently, a research on the stem cells is competitively in progress around the world. If a technology in which humans can appropriately control the stem cells is secured, organ transplantation may be very easily made, and as a result, a new world of disease treatment will be opened.

In recent years, an active stem cell transporter methodology has been presented, which accurately guides a microrobot with the stem cells to a damaged cartilage portion in an electromagnetic field control method for regeneration of the articular cartilage. Such a method is expected to be in the limelight as a new treatment method to remarkably reduce treatment cost while shortening a recovery period of a patient and increasing a treatment effect depending on active driving as compared with an existing low-efficiency non-invasive stem cell injection method at the time of treating the articular cartilage which is a representative degenerative disease region.

FIG. 1 is a diagram illustrating a microscope image of a microrobot with stem cells in the related art.

The microrobot with the stem cells in the related art illustrated in FIG. 1 is fabricated as a three-dimensional porous structure consisting of a biodegradable polymer, in which nano magnetic particles responding to an external magnetic field are attached on a surface thereof, and the stem cells are mounted in the self-driving microrobot.

The microrobot with the stem cells may be precisely moved to a damaged articular cartilage area according to a response of the nano magnetic particles on the microrobot by the external magnetic field. After the microrobot is moved, the stem cells are differentiated into cartilage cells and simultaneously, the microrobot is gradually decomposed in the body.

When briefly describing a manufacturing process of the microrobot with the stem cells illustrated in FIG. 1, a magnetic body (diameter of 5 nm to 100 nm) is added to the cells and then co-cultured for 12 hours to manufacture cells containing the magnetic body responding to a magnetic field in the cells by the action of endocytosis which introduces a material into the inside from the outside using a cell membrane. At this time, a spheroid-shaped cell structure may be manufactured by a method of three-dimensionally culturing the cells and the magnetic body in a microstructure such as a scaffold. Here, a PLGA micro-scaffold body is fabricated by a PLGA double emulsion method, polyethylenimine (PEI) coated with Fe₃O₄MNP is attached on the surface of the PLGA micro-scaffold fabricated using a coupling process using amino bond formation to fabricate a micro-scaffold attached with magnetic nano-particles. The fabricated micro-scaffold is immersed in a DMEM medium containing 10% FBS, the stem cells are injected on the micro-scaffold under a microscope, and then cultured in an incubator to mount the stem cells in the micro-scaffold.

Meanwhile, as illustrated in FIG. 2, an existing medium-animal target pre-clinical cell mounting method had a limitation to be used in an actual treatment environment using a large-capacity spinner flask.

FIG. 2 is a diagram illustrating a medium-animal target pre-clinical cell mounting method in the related art.

Referring to FIG. 2, in the medium-animal target pre-clinical, a three-necked spinner flask, a flex-type/bulb-shaped magnetic glass, a rotary impeller assembly, a teflon/silicon-based screw cap, and the like are used. Here, a rotary shaft is connected to a “stirring shaft-ring” attached to a center in the flask cap above a sample and the rotary shaft assembly is constituted by a special bulb-shaped glass magnetic impeller. The shaft impeller is a length adjustable type, in which the glass bulb impeller rotates (stirs) at a low speed around the bottom of the flask by a magnetic stirrer.

Existing commercial flasks have a large-capacity of 125 ml or larger, which cannot be applied to a small-sized capacity, and thus there is a problem in that a volume is large and a lot of cost is required, such as consuming a large amount of consumable materials.

Further, an external power supply is required, there is a possibility of cross-infection due to an increase in mounting time of 3 hours or more and repeated uses, and thus cleaning and sterilization processes are required for each time, and there is a possibility of microorganism infection during the mounting of the therapeutic agent, and thus there is a problem that it is difficult to use the existing commercial flask in a medical environment.

### DISCLOSURE

### Technical Problem

An embodiment of the present invention is to provide a cell mounting module device which is fabricated in a small-sized module form to improve cell mounting and recovery efficiency for a microstructure and exclude a risk of cross-contamination in a disposable use.

Another embodiment of the present invention is to provide a cell mounting module device capable of mounting a large possible amount of stem cells on a polymeric structure so as to transfer therapeutic stem cells efficiently into body lesions.

### Technical Solution

In embodiments, a cell mounting module device includes a body portion having a container form of which upper and lower portions are opened and an inside is empty, a cap portion which includes an upper cap and a lower cap coupled to opened upper and lower ends of the upper and lower portions of the body portion to be opened and closed, respectively, and has a syringe coupling member formed at a center of the lower cap, a cell accommodating portion which is installed in the body portion and accommodates cells injected through the opened upper portion of the body portion, and a cell mounding module portion which is installed below the cell accommodating portion and includes a microstructure for mounting the cells accommodated in the cell accommodating portion.

The body portion may be formed of a cylindrical can body and fabricated in a small size to be suitable for a small capacity.

The cell mounting module portion may include a microstructure, and a fluid channel and a fluid valve channel connecting the microstructures, which are configured by patterning based on a microfluid control.

The microstructure as a three-dimensional porous structure made of a biodegradable polymer may have a form in which nano magnetic particles responding to an external magnetic field are attached onto the surface.

The cell accommodating portion may have an inclined surface to collect the cells injected into the body portion at the center and an opening formed on a bottom surface, through which the cells may pass, and the cells may pass through the opening in a moving direction of a piston of the syringe fastened to the syringe coupling member to move to the microstructure.

The cell mounting module portion may further include a filter member which is disposed below the microstructure and filters the cells passing through the opening of the cell accommodating portion to concentrate the cells at a high concentration and enhance cell mounting efficiency of the microstructure.

The cell mounting module device may further include an air filter which is installed in the upper cap and filters air introduced into the body portion to prevent contamination.

The cells may be stem cells.

### Advantageous Effects

The disclosed technology may have the following effects. However, since as it is not meant that a particular embodiment should include all of the following effects or merely include the following effects, it should be understood that the scope of the disclosed technology is not to be construed as being limited thereby.

According to the embodiment of the present invention, the cell mounting module device is fabricated in a small-sized module form to be suitable for a small-scale capacity, can be used in a disposable use to exclude a possibility of cross-infection, and may maintain a cell culture environment while the cells are attached to the microstructure to prevent contamination.

According to the embodiment of the present invention, the cell mounting module device may mount the cells on the microstructure without a separate power source due to generation of power using a syringe.

According to the embodiment of the present invention, in the cell mounting module device, a fine filter is disposed so the cells may be filtered to concentrate the cells at a high concentration, and a large possible amount of cells are attached to the microstructure to improve the cell mounting and recovery efficiency.

### DESCRIPTION OF DRAWINGS

FIG. 1 is a diagram showing a microscopic image of a microrobot with stem cells in the related art.

FIG. 2 is a diagram illustrating a medium-animal target pre-clinical cell mounting method in the related art.

FIG. 3 is a diagram illustrating a cell mounting module device according to an embodiment of the present invention.

FIG. 4 is an exemplary diagram illustrating a cell mounting module portion of FIG. 3.

FIG. 5 is a diagram illustrating a fastening state between the cell mounting module device of FIG. 3 and a syringe.

FIG. 6 is a schematic diagram illustrating the cell mounting module device of FIG. 3.

FIG. 7 is a diagram illustrating a cell mounting method of the cell mounting module device of FIG. 6.

FIG. 8 is a diagram illustrating a cell recovering method of the cell mounting module device of FIG. 7.

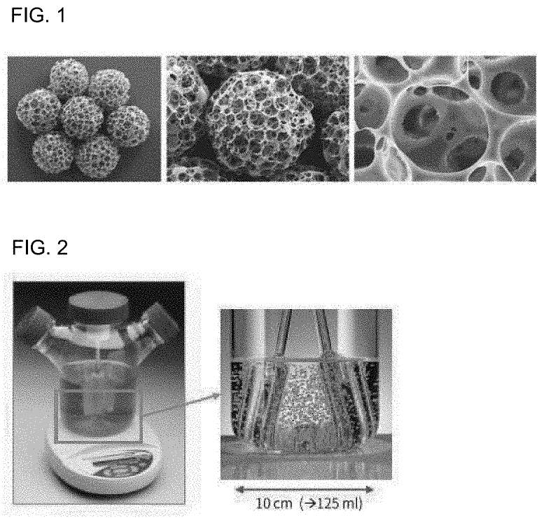

### BEST MODE FOR INVENTION

A best aspect of the present invention provides a cell mounting module device comprising a body portion having a container form of which upper and lower portions are opened and an inside is empty, a cap portion which includes an upper cap and a lower cap coupled to opened upper and lower ends of the upper and lower portions of the body portion to be opened and closed, respectively, and has a syringe coupling member formed at a center of the lower cap, a cell accommodating portion which is installed in the body portion and accommodates cells injected through the opened upper portion of the body portion, and a cell mounding module portion which is installed below the cell accommodating portion and includes a microstructure for mounting the cells accommodated in the cell accommodating portion.

### MODE FOR INVENTION

A description of the present invention is merely an embodiment for a structural or functional description and the scope of the present invention should not be construed as being limited by an embodiments described in the specification. That is, since the embodiment can be variously changed and have various forms, the scope of the present invention should be understood to include equivalents capable of realizing the technical spirit. Further, it should be understood that since a specific embodiment should include all objects or effects or include only the effect, the scope of the present invention is limited by the object or effect.

Meanwhile, meanings of terms described in the present application should be understood as follows.

The terms “first,” “second,”, and the like are used to differentiate a certain component from other components, but the scope of should not be construed to be limited by the terms. For example, a first component may be referred to as a second component, and similarly, the second component may be referred to as the first component.

It should be understood that, when it is described that a component is “connected to” another component, the component may be directly connected to the other component or a third component may be present therebetween. In contrast, it should be understood that, when it is described that an element is “directly connected to” another element, it is understood that no element is present between the element and another element. Meanwhile, other expressions describing the relationship of the components, that is, expressions such as “between” and “directly between” or “adjacent to” and “directly adjacent to” should be similarly interpreted.

It is to be understood that the singular expression encompass a plurality of expressions unless the context clearly dictates otherwise and it should be understood that term “include” or “have” indicates that a feature, a number, a step, an operation, a component, a part or the combination thereof described in the specification is present, but does not exclude a possibility of presence or addition of one or more other features, numbers, steps, operations, components, parts or combinations thereof, in advance.

If it is not contrarily defined, all terms used herein have the same meanings as those generally understood by those skilled in the art. Terms which are defined in a generally used dictionary should be interpreted to have the same meaning as the meaning in the context of the related art, and are not interpreted as an ideal meaning or excessively formal meanings unless clearly defined in the present application.

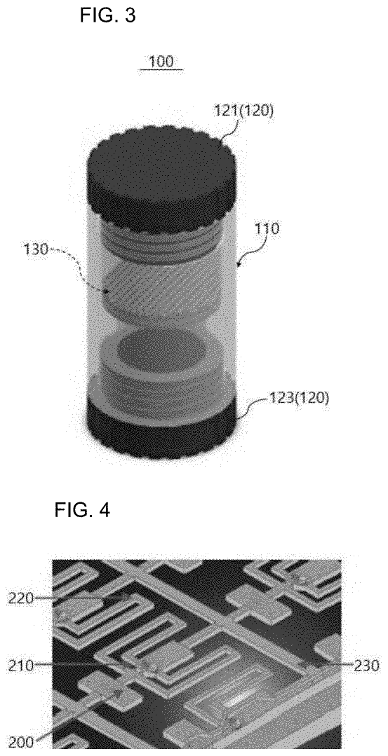

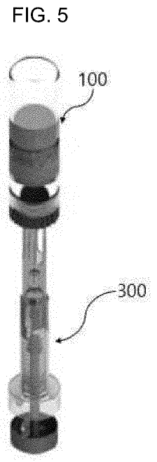

FIG. 3 is a diagram illustrating a cell mounting module device according to an embodiment of the present invention, FIG. 4 is an exemplary diagram illustrating a cell mounting module portion of FIG. 3, and FIG. 5 is a diagram illustrating a fastening state between the cell mounting module device of FIG. 3 and a syringe.

Referring to FIGS. 3 to 5, a cell mounting module device 100 is fabricated in a small size of approximately 10×5 cm or less and may be formed of a cylindrical-shaped transparent plastic material. In one embodiment, the cell mounting module device 100 is suitable for a small capacity of 10 ml or less and may be fabricated in a disposable use.

In one embodiment, the cell mounting module device 100 may include a body portion 110, a cap portion 120, and a cell mounting module portion 130.

The body portion 110 is fabricated in a small size in a substantially cylindrical can body as a container of which upper and lower portions are opened and the inside is empty. In the body portion 110, the cap portion 120 is coupled to opened upper and lower ends of the upper and lower portions to be opened and closed. The cell mounting module portion 130 is fixed and installed in the body portion 110.

The cap portion 120 is constituted by an upper cap 121 and a lower cap 123, which are coupled to the opened upper and lower portions of the body portion 110 to be opened and closed by turning, respectively. Here, the cap portion 120 serves as a cap of the body portion 110.

The upper cap 121 is opened to open the upper portion of the body portion 110 when the cells are injected into the body portion 110 and the cells are recovered.

In one embodiment, the lower cap 123 may include a syringe coupling member for coupling with the syringe 300. Here, the syringe coupling member may be formed in a structure through which a tip portion of the syringe 300 may pass.

In one embodiment, the cell mounting module portion 130 may be fabricated as a module by using a soft lithography technique and may include a microstructure 210, and a fluid channel 220 and a fluid valve channel 230 connecting respective structures 210, which are configured by patterning the microfluid as illustrated in FIG. 4.

The soft lithography refers to a molding method using a silicon elastomer used n semiconductor manufacturing. In the semiconductor industry, the soft lithography is mainly used for the production of chips of nanometer sizes in micrometers, but for medicine or medical treatment, the soft lithography is used to be cheaply used in a medical field from cytological studies to diagnosis and treatment by mass-producing chips as a molding article of a millimeter size regardless of a size.

The soft lithography technique has been used for formation of microfluid patterns, and for example, since a microfluid chip made of polydimethyl siloxane (PDMS) is a polymer, the microfluid chip is good in flexibility and excellent in biocompatibility for cells and biomaterials, and the surface may be chemically easily reformed to introduce a required chemical functional group.

The microfluid may have many characteristics different from a general fluid. The microfluid has a laminar flow characteristic having the Reynolds number of 2000 or less. The Reynolds number (Re) is calculated through the following Equation.

Re=ρvd/μ [Equation]

Here, ρ represents a fluid density (g/cm³), v represents a fluid velocity (cm/s), d represents a diameter (cm) of the channel, and μ represents the viscosity (g/cm·s).

In this condition, the flow of the fluid is primarily dependent on the viscosity. In the microfluid, when two types of fluids flow while being in contact with each other, the two fluids are mixed only by diffusion on a contact surface to obtain a stable concentration gradient. In a cell assay system based on the microfluid, various types of cells or tissues may be implanted in a micro chamber, nutrients may be continuously supplied to the chamber using a micro channel, and by-products generated in the cell metabolism may be removed. A small-sized device is used to maintain an advantage of reducing an amount of biochemical drugs used for cell samples and cell culture.

In one embodiment, the cell mounting module portion 130 may mount the stem cells 200 injected into the body portion 110 on the microstructure 210 based on a fluid control and using a piston operation of the syringe 300 as a power source as illustrated in FIG. 5. Here, the stem cells 200 may be injected through the upper portion of the body portion 110 which is opened by opening the upper cap 121.

Referring to FIG. 5, after the syringe 300 is coupled to the syringe coupling member of the lower cap 123, when a piston of the syringe 300 is pulled downwardly, the stem cells 200 injected to the upper portion by the pressure move to the lower portion to be mounted on the microstructure 210.

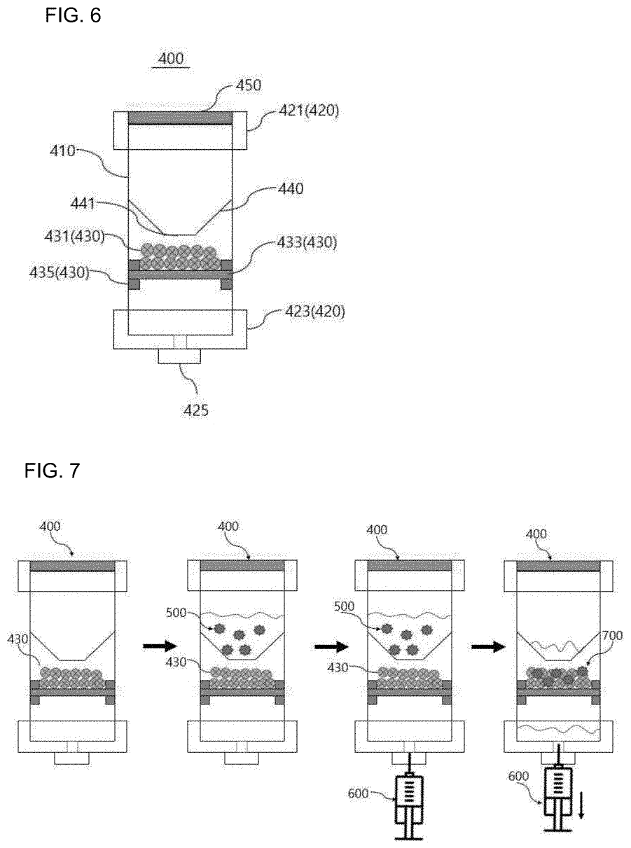

FIG. 6 is a schematic diagram illustrating the cell mounting module device of FIG. 3.

Referring to FIG. 6, a cell mounting module device 400 may include a body portion 410, a cap portion 420, a cell mounting module portion 430, a cell accommodating portion 440, and a filter portion 450.

The body portion 410 is formed of a small-sized transparent material as a hollow cylindrical can body and serves as a case for protecting the cell mounting module portion 430. The body portion 410 has opened upper and lower portions and the cap portion 420 is coupled to the opened upper and lower ends to be opened and closed. The body portion 410 is installed with the cell mounting module portion 430 therein.

The cap portion 420 is constituted by an upper cap 421 and a lower cap 423, which are coupled to the body portion 410 to be opened and closed by turning. The lower cap 423 is provided with a syringe coupling member 425 in the center thereof. The syringe coupling member 425 may be formed in a structure which is able to be fastened with a syringe 600. In one embodiment, the syringe coupling member 425 may be fastened to the syringe 600 so that a tip portion of the syringe 600 is inserted through a through-hole by forming the through-hole between the lower cap 423 and the lower end of the body portion 410.

The cell mounting module portion 430 includes a microstructure 431, a filter member 433, and a filter fixing member 435.

The microstructure 431 as a three-dimensional porous structure made of a biodegradable polymer is fabricated in the form where nano magnetic particles responding to an external magnetic field are attached onto the surface thereof, and mounted with the cells.

The filter member 433 is disposed below the microstructure 431 to filter the cells so that the cells are concentrated at a high concentration.

The filter fixing member 435 is installed around an inner peripheral surface of the body portion 410 and may fix the filter member 433 at both sides thereof.

The cell accommodating portion 440 has an inclined surface to collect the cells injected into the body portion 410 at the center and an opening 441 formed on a bottom surface, through which the cells may pass.

The filter portion 450 is installed on the upper cap 421 and may be implemented as an air filter to prevent contamination when air is introduced into the body portion 410.

As such, the cell mounting module device 400 fabricated in a small size may inject the cells 500 in an actual medical field and may be mounted on the microstructure 431. In one embodiment, the cells 500 may be stem cells.

FIG. 7 is a diagram illustrating a cell mounting method of the cell mounting module device of FIG. 6.

Referring to FIG. 7, the cells 500 are injected into the cell mounting module device 400. In one embodiment, the upper cap 421 of the cell mounting module device 400 is turned to open the upper portion of the body portion 410 and then the cells 500 may be injected. At this time, the cells 500 injected into the body portion 410 are accommodated in the cell accommodating portion 440. Here, the cells 500 may be included in the culture solution and injected and collected at the center along the inclined surface formed on the cell accommodating portion 440. At this time, the cells 500 collected in the cell accommodating portion 440 are accommodated in the cell accommodating portion 440 without passing through the opening 441 formed in the bottom surface by the surface tension.

Thereafter, cell mounting power is generated using the syringe 600 in the cell mounting module device 400. In one embodiment, after the syringe 600 is fastened to the syringe coupling member 425 of the lower cap 423, the power is generated in the body portion 410 by a pumping operation. At this time, the syringe 600 allows the piston to move outward based on the cell mounting module device 400 to generate a pressure (force) in the moving direction of the piston in the body portion 410 of the cell mounting module device 400. Accordingly, the cells 500 accommodated in the cell accommodating portion 440 are attached to the microstructure 431 through the opening 441 provided at the bottom of the cell accommodating portion 440 to fabricate a cell mounting microstructure 700. At this time, the filter member 433 passes the culture solution therethrough and filters the cells 500 so that the cells 500 are concentrated at a high concentration, thereby enhancing the cell mounting efficiency of the microstructure 431. Here, the filter portion 450 installed in the upper cap 421 may filter contaminated air which may be introduced while a pressure (force) is generated in the body portion 410 by a piston operation of the syringe 600. Accordingly, it is possible to minimize contamination during the cell mounting and a possibility of microorganism infection.

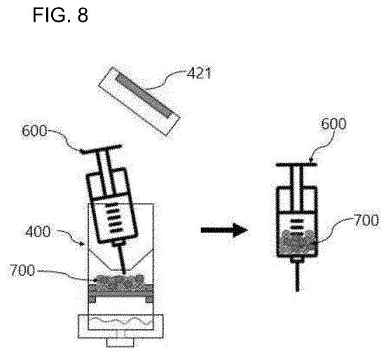

FIG. 8 is a diagram illustrating a cell recovering method mounted on the microstructure of FIG. 7.

Referring to FIG. 8, the upper cap 421 is turned to open the upper portion of the body portion 410 and then a needle portion of the syringe 600 passes through the opening 441 of the cell accommodating portion 440 by the opened upper portion. Thereafter, the piston of the syringe 600 is pulled outward to generate the power. The cell mounting microstructure 700 is introduced into the syringe 600 along the needle in the moving direction of the piston of the syringe 600 to recover the stem cells mounted on the cell mounting microstructure 700.

According to an embodiment, the cell mounting module device does not require an external power supply, is suitable for a small capacity of 10 and is possible in a disposable use to exclude a possibility of cross infection. In addition, the cell mounting module device can be sterilized and separately packaged in a small size of 10×5 cm or less and may expect high cell mounting and recovery efficiency as compared with the related art by controlling a fluid flow through a pressure control and applying a fine filter to be anticipated to expand the use of the actual medical field.

The present invention has been described with reference to the preferred embodiments, but those skilled in the art will understand that the present invention can be variously modified and changed without departing from the spirit and the scope of the present invention which are defined in the appended claims.

### NATIONAL RESEARCH AND DEVELOPMENT PROJECT SUPPORTING PRESENT INVENTION

Project Unique Number: 2017M3A9E9075572

Department Name: Ministry of Science and ICT

Research Management Organization: National Research Foundation of Korea

Research Project Name: Development of Bio. Medical Technology

Research Subject Name: Development of Cartilage Regeneration Substitutes Using Microstructure Targeting Technology and Construction of Customized Support System for

Commercialization

Percent Contribution: 111

Managing Department: Chonnam National University Hwasun Hospital

Research Period: Nov. 1, 2016 to Jul. 31, 2021

### EXPLANATION OF REFERENCE NUMERALS AND SYMBOLS

100,400: Cell mounting module device
110,410: Body portion
120,420: Cap portion
121,421: Upper cap
123,423: Lower cap
130,430: Cell mounting module portion
200,500: Cell
210,431: Microstructure
220: Fluid channel
230: Fluid valve channel
300,600: Syringe
425: Syringe coupling member
433: Filter member
435: Filter fixing member
440: Cell accommodating portion
441: Opening
450: Filter portion
700: Cell mounting microstructure

### INDUSTRIAL AVAILABILITY

As described above, the cell mounting module device according to the present invention may be used in a medical environment such as a stem cell research and a stem cell implantation surgery for disease treatment.

1. A cell mounting module device comprising: a body portion having a container form of which upper and lower portions are opened and an inside is empty;
a cap portion which includes an upper cap and a lower cap coupled to opened upper and lower ends of the upper and lower portions of the body portion to be opened and closed, respectively, and has a syringe coupling member formed at a center of the lower cap;
a cell accommodating portion which is installed in the body portion and accommodates cells injected through the opened upper portion of the body portion; and
a cell mounding module portion which is installed below the cell accommodating portion and includes a microstructure for mounting the cells accommodated in the cell accommodating portion.

2. The cell mounting module device of claim 1, wherein the body portion is formed of a cylindrical can body and fabricated in a small size to be suitable for a small capacity.

3. The cell mounting module device of claim 1, wherein the cell mounting module portion includes a microstructure, and a fluid channel and a fluid valve channel connecting the microstructures, which are configured by patterning based on a microfluid control.

4. The cell mounting module device of claim 1, wherein the microstructure as a three-dimensional porous structure made of a biodegradable polymer has a form in which nano magnetic particles responding to an external magnetic field are attached onto the surface.

5. The cell mounting module device of claim 1, wherein the cell accommodating portion has an inclined surface to collect the cells injected into the body portion at the center and an opening formed on a bottom surface, through which the cells may pass, and the cells passes through the opening in a moving direction of a piston of the syringe fastened to the syringe coupling member to move to the microstructure.

6. The cell mounting module device of claim 5, wherein the cell mounting module portion further includes a filter member which is disposed below the microstructure and filters the cells passing through the opening of the cell accommodating portion to concentrate the cells at a high concentration and enhance cell mounting efficiency of the microstructure.

7. The cell mounting module device of claim 1, further comprising:
an air filter which is installed in the upper cap and filters air introduced into the body portion to prevent contamination.

8. The cell mounting module device of claim 1, wherein the cells are stem cells.
